(19) United States
(12) Patent Application Publication
(10) Pub. No.: US 2020/0025721 A1
(43) Pub. Date: Jan. 23, 2020

(54) Analytical Laboratory Testing Procedures

(71) Applicant: Prompt Praxis Laboratories LLC, Vernon Hills, IL (US)

(72) Inventors: Lisa McChesney, Vernon Hills, IL (US); Cecilia Newcomb, Vernon Hills, IL (US)

(21) Appl. No.: 16/519,256

(22) Filed: Jul. 23, 2019

Related U.S. Application Data
(60) Provisional application No. 62/701,894, filed on Jul. 23, 2018.

(51) Int. Cl. G01N 30/06; G16H 10/40

(52) U.S. Cl. CPC G01N 30/06; G01N 33/53; G16H 10/40

(57) ABSTRACT

A test method developed with the goals of rapid turnaround, short run times (target under 5 minutes), small volumes for dilution (target 10 mL), fewest number of mobile phases, rapid transition between analyses, minimized opportunity for preparation errors, leverage Autoblend® function of equipment, easily maintain on-hand solution inventory, and avoiding ion-pairing agents and other additives. The test method development strategy begins with vendor application notes, USP, and literature references. The strategy targets families of chemically similar compounds, whenever possible, and leverages new column technologies. Finally, the strategy standardizes validation and sample preparation procedures.

### RELATED APPLICATION

The present application claims the filing priority of U.S. Provisional Application No. 62/701,894 titled “Analytical Laboratory Testing Procedures” filed on Jul. 23, 2018. The entirety of the '894 application is hereby incorporated by reference.

### TECHNICAL FIELD OF THE INVENTION

The present invention relates to methods for performing analytical laboratory tests.

### BACKGROUND OF THE INVENTION

There are numerous reasons for the need of accurate analytical testing. There are also numerous reasons for rapid analytical testing. In many instances, due to the inverse relationship between accuracy and speed, one of these needs is usually sacrificed for the benefit of the other. However, as techniques and technology advance, the necessity of getting accurate analytical results need not come at the expense of time, and vice versa.

Applicants have developed very specific procedures which allow increased speed in analytical testing without sacrificing accuracy of results.

Until the invention of the present application, these and other problems in prior art testing procedures went either unnoticed or unsolved by those skilled in the art. The present inventive methods provide solutions for saving time without sacrificing testing accuracy, reproducibility, or creating error.

### SUMMARY OF THE INVENTION

There are disclosed herein, improved analytical testing procedures which avoid the disadvantages of prior procedures and methods, while affording additional time and accuracy advantages.

Generally speaking, the invention is directed to a liquid chromatography testing method for reducing laboratory waste and testing times and comprises the steps of accepting a plurality of samples to be tested on a liquid chromatograph, preparing at least one analyte for each of the plurality of samples to create a plurality of test analytes, utilizing ultra-high-performance liquid chromatography, selecting autosampler vials to allow analysis of as little as 300 microliters of sample, grouping analytes by families to allow a single chromatographic column to be used for all analytes in a family, running a combination standard for multiple chemicals on the liquid chromatograph, and creating a subset of the plurality of test analytes based on those containing at least one of the multiple chemicals in the combination standard.

In a preferred embodiment, the step of preparing at least one analyte comprises the steps of limiting a volumetric size of laboratory glassware used to no more than 10.0 mL, and limiting transfer of liquids to be no more than 100 microliters.

In an embodiment of the disclosed method, the families of analytes are selected from the group comprising CII-CIV controlled substances, the Caine family, the Catacholamine-like family, Reversal Agents, and the Cardiac family. Accordingly, the organic portion of the mobile phase would be adjusted while the buffer potion-portion of the mobile phase would remain the same for all analytes in a family.

These and other aspects of the invention may be understood more readily from the following description and the appended drawings, tables and other documents.

### BRIEF DESCRIPTION OF THE DRAWINGS

For the purpose of facilitating an understanding of the subject matter sought to be protected, there are illustrated in the accompanying drawings, embodiments, components and results thereof, from an inspection of which, when considered in connection with the following detailed description, the subject matter sought to be protected, its construction and operation, and many of its advantages should be readily understood and appreciated by those of skill in the relevant art.

FIG. 1 is a flow chart showing an embodiment of the analytical procedure at a high level.

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

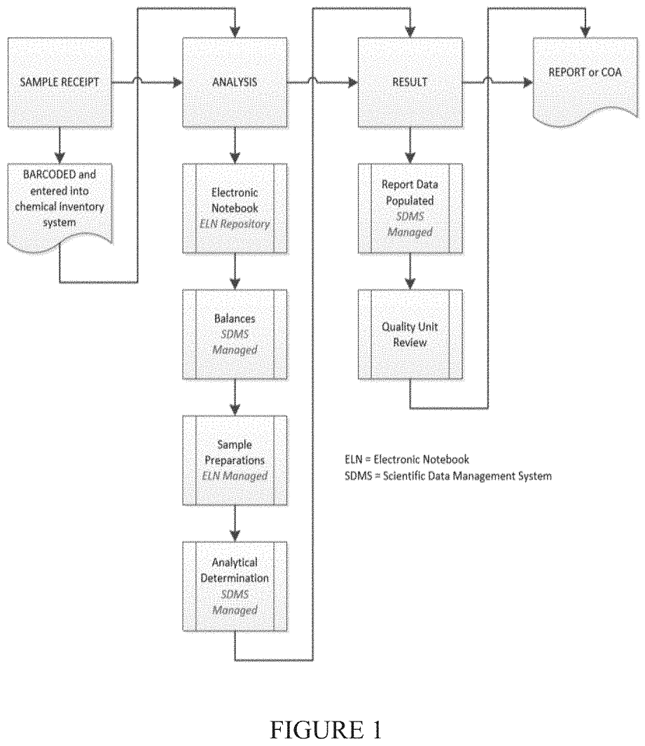

Referring to FIG. 1, there is illustrated aspects of the disclosed test procedures. The disclosed test method was developed with the goals of rapid turnaround, short run times (target under 5 minutes), small volumes for dilution (target 10 mL), fewest number of mobile phases, rapid transition between analyses, minimized opportunity for preparation errors, leverage Autoblend® function of equipment, easily maintain on-hand solution inventory, and avoiding ion-pairing agents and other additives.

The test method development strategy began with vendor application notes, USP, and literature references. The strategy targeted families of chemically similar compounds, whenever possible, and leveraged new column technologies. Finally, the strategy standardized validation and sample preparations procedures.

Application of the improved analytical procedure included creating a library of reference chromatograms, rapid turnaround, synergistic analyses, fast transition between test methods, significantly reduced solvent waste stream, and uniquely identify a drug product from within a family thus quickly identifying any admixture mix-ups. TABLE 1 below shows a list of substances which can be analyzed for product strength, quality, purity, potency and identity.

TABLE 1

| | | | |
|---|---|---|---|
| Adenosine | Amiodarone | Articaine | Atropine |
| Bupivacaine | Cefazolin | Cisatracurium | Dexamethasone Sodium Phosphate |
| Dexmedetomidine | Diltiazem | Dobutamine | Dopamine |
| Ephedrine | Epinephrine | Esmolol | Famotidine |
| Fentanyl CII | Fentanyl CII/Bupivacaine | | Fentanyl CII/Ropivacaine |
| Furosemide | Glycopyrrolate | Granisetron | Heparin-(Bioassay) |
| Hydromorphone CII | Ketamine CIII | Ketorolac | Labetalol |
| Lidocaine | Lorazepam CIV | Meperidine CII | Mepivacaine |
| Methadone CII | Methohexital CIV | Metoclopramide | Metoprolol |
| Midazolam CIV | Milrinone | Morphine CII | Nalbuphine |
| Neostigmine | Nicardipine | Nitroglycerin | Norepinephrine |
| Ondansetron | Oxytocin (conc) | Phenylephrine | Prilocaine |
| Procaine | Propofol | Propranolol | Ranitidine |
| Remifentanil CII | Rocuronium | Ropivacaine | Succinycholine |
| Sufentanil CII | Sufentanil CII/Bupivacaine | | Sufentanil CII/Ropivacaine |
| Terbutaline | Tetracaine | Theophylline | Vancomycin |
| Vasopressin | Vecuronium | | |

There are three basic design factors have created the present platform and highly efficient workflows:

1) Miniaturize everything possible;

2) Develop methods around families of analytes; and

3) Fuse IT functionality with lab capabilities.

As to the first design factor, 10.0 mL volumetric flasks are used—it takes substantially less time and materials to fill a 10 mL volumetric flask (VF) than a 50 or 100 mL VF. Also, highly precise, small volume transfer pipets on the order of 50 to 100 microliter transfers are used. The sample volume is reduced and easily transferred in very rapid fashion to the 10.0 mL VF. The pipet tips are disposable and thus save time/energy by eliminating both the need to clean glass volumetric pipets, and the concern about cross-contamination. Finally, the use of ultra high-performance liquid chromatograph (uPLC) technology requires much less volume on column for analysis (2-5 microliters instead of 10-25 microliter) and the autosampler viaN selected can allow analysis with as little at 300 microliters of sample.

As to the second design factor, the disclosed testing procedure takes advantage of commonalities found in families of analytes. By way of example, chromatography identified for Fentanyl (a controlled substance) in the literature was successfully applied to the family of all CII-CIV controlled molecules, including Hydromorphone, Ketamine, Lorazepam, Meperidine, Methadone, Midazolam, Morphine, Remifentanil, and Sufentanil. Thus, all of these molecules can be tested on a single uPLC and chromatographic column and the time between molecules to change over is only about seven (7) minutes without the need for cleaning between samples. Specifically, the buffer used in the mobile phase can remain the same for all molecules and the organic portion can be lowered or increased to facilitate the analysis.

This same approach was adopted for the cardiac family of molecules, which include Amiodarone, Diltiazem, Esmolol, Labetalol, Metoprolol, Nicardipine, and Propranolol. As with the above CII-CIV molecules, all of these cardiac family molecules can be tested on a single uPLC and chromatographic column. Likewise, the change over time between molecules is also only about seven (7) minutes without the necessity of cleaning between analytes. Again, the buffer should remain the same for all molecules and the organic portion should be lowered or increased to facilitate the analysis.

Other grouping of samples may include the Antihistamines, Antiemetics, Caine Family, the Catacholamine-like family, Reversal Agents, and Similar molecules—such as Atropine, Neostigmine and Glycopyrrolate. Antihistamines include Famotidine and Ranitidine and Antiemetics include Granisetron, Metoclopramide, and Ondansetron. Caine Family substances include Bupivacaine, Lidocaine, Mepivacaine, Prilocaine, Procaine, Ropivacaine, Tetracaine, Fentanyl-Bupivacaine combination test articles, Fentanyl-Ropivacaine combination test articles, Sufentanil-Bupivacaine combination test articles, and Sufentanil-Ropivacaine combination test articles. The Catacholamine Like Family includes Dobutamine, Dopamine, Epinephrine, Norepinephrine, and Phenylephrine. Reversal Agents include Rocuronium, Pancuronium, and Vecuronium. All of the above scenarios minimize the waste stream volumes for our systems.

Using this analyte grouping step, applicants were able to analyze 18 different molecules on a single day within an 8-hour period.

To expand a bit further on this efficiency step, combination standards can be used to further save time and reduce waste. That is, if the chromatography is the same, then a combination standard can be run at the beginning of the day, and all samples that contain one or more of the analytes, can be subsequently analyzed without interruption. For example, a combination standard containing Lidocaine, Ropivacaine, Bupivacaine, Fentanyl and Sufentanil, can be performed, then all samples containing at least one of these analytes can be tested against the respective standard. Time is saved by only having to perform a single set of system suitability injections and not five sets of system suitability injections. As families of molecules are analyzed with very similar parameters, if a test article is mislabeled, it would immediately become evident by virtue of knowing the elution order of all molecules in a family. Further, through use of a Library of Reference Chromatograms, one can quickly identify and apply existing test methodology to structurally similar new molecules while maintaining the rapid transition paradigm and consistent application of the same buffer/column systems.

Finally, as to the third efficiency factor, the use of IT functionality is used and integrated with lab capabilities whenever possible. For example, data from ancillary equipment is captured by electronic means, such as standard weights, pH values, moisture values, and this data is then archived and verified by a Quality Assurance group. Further, all laboratory work is captured in electronic laboratory notebooks (ELNs) so that it can be easily cloned and systematized. This facilitates streamlining workflows and can be made immediately visible and accessible to the

Quality Assurance group.

The disclosed testing procedures are especially applicable to the testing listed in TABLE 2 below:

TABLE 2

| | Analytical Development | Research & |
|---|---|---|
| Analytical Testing | and Validation Services | Development |
| Batch Level Release Testing | Stability Indicating Assay | Assist in Proposing |
| | | Drug Product Specs |
| Heparin Potency | Related Substance/Impurity | Container Closure |
| | | Selection & Evaluation |
| Complaints/Investigation Testing | Pre-formulation Support Testing | Compatibility Studies |
| Third Party Confirmation Testing | Residual Solvent Analyses | ICH Compliant |
| | | StabilityStudies |
| R&D Stability Testing | Remedial Validation | CMC Support |
| Identification Testing | Gap Analysis | |
| Moisture Determination | Process Validation Support | |
| Water Vapor Transmission Testing | Method Transfer Support | |
| Melting Point Determination | Formulation Development Support | |
| Specific Gravity Determination | Comparative Studies | |
| Osmolality Determination | Forced Degradation Studies | |
| Optical Rotation Determination | Light Stressing of Sample per ICH | |
| FTIR Identification Testing | | |
| Heavy Metals by ICP-OES | | |
| UV/Vis Spectroscopy | | |
| Color Determination vs. Hunterlab | | |
| Globule Size Analysis | | |
| Particulate Matter Determination | | |
| BET Testing | | |
| Validated Test Method Licensing | | |

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A liquid chromatography testing method for reducing laboratory waste and testing times, the method comprising the steps of:
accepting a plurality of samples to be tested on a liquid chromatograph;
preparing at least one analyte for each of the plurality of samples to create a plurality of test analytes, wherein the step of preparing at least one analyte comprises the steps of:
limiting a volumetric size of laboratory glassware used to no more than 10.0 mL;
limiting transfer of liquids to be no more than 100 microliters;
utilizing ultra-high-performance liquid chromatography;
selecting autosampler vials to allow analysis of as little as 300 microliters of sample;
grouping analytes by families to allow a single chromatographic column to be used for all analytes in a family;
running a combination standard for multiple chemicals on the liquid chromatograph; and
creating a subset of the plurality of test analytes based on those containing at least one of the multiple chemicals in the combination standard.

2. The method of claim 1, wherein the families of analytes are selected from the group comprising Antihistamines, Antiemetics, CII-CIV controlled substances, the Caine family, the Catacholamine-like family, Reversal Agents, and the Cardiac family.

3. The method of claim 2, further comprising the step of adjusting an organic portion of a mobile phase.

4. The method of claim 3, wherein the buffer potion of the mobile phase remains the same for all analytes in a family.

5. The method of claim 1, further comprising the step of storing all test data in an electronic laboratory notebook.

6. An analytical testing procedure comprising the steps of:
receiving a plurality of samples to be analyzed;
grouping the plurality of samples by chemical family;
preparing test samples for each of the plurality of samples comprising the step of transferring no more than 100 microliters of each sample to an autosampler vial to create a plurality of test samples;
analyzing each of the plurality of test samples by family groupings, comprising the steps of:
running test samples through an ultra high-performance liquid chromatograph;
capturing data from the ultra high-performance liquid chromatograph in an electronic notebook;
reviewing captured data in electronic notebook for anomalies;
reporting results of testing.

7. The analytical testing procedure of claim 6, wherein the family groupings are selected from the group comprising Antihistamines, Antiemetics, CII-CIV controlled substances, the Caine family, the Catacholamine-like family, Reversal Agents, and the Cardiac family.

8. The analytical testing procedure of claim 7, further comprising the step of adjusting an organic portion of a mobile phase.

9. The analytical testing procedure of claim 8, wherein the buffer potion of the mobile phase remains the same for all analytes in a family.
